(19) United States
(12) Patent Application Publication
(10) Pub. No.: US 2012/0115745 A1
(43) Pub. Date: May 10, 2012

(54) METHODS FOR PREDICTING SENSITIVITY TO TREATMENT WITH A TARGETED TYROSINE KINASE INHIBITOR

(76) Inventors: Evelyn M. McKeegan, Lake Forest, IL (US); Peter Ansell, Grayslake, IL (US); Barry L. Dowell, Mundelein, IL (US); Ke Zhang, Grand Forks, ND (US); Viswanath Devanarayan, Souderton, PA (US); Arunava Charkravartty, Wheeling, IL (US)

(21) Appl. No.: 13/292,959

(22) Filed: Nov. 9, 2011

Related U.S. Application Data
(63) Continuation-in-part of application No. 13/100,869, filed on May 4, 2011, now abandoned.
(60) Provisional application No. 61/332,545, filed on May 7, 2010.

(51) Int. Cl. C40B 30/04; C40B 40/10; G01N 33/566

(52) U.S. Cl. 506/9; 435/7.1; 436/501; 435/7.4; 506/18

(57) ABSTRACT

The present disclosure relates generally to the evaluation and/or treatment of a subject having or suspected of having a neoplastic condition, and in particular to the use of biomarkers for identifying patients receptive to a certain drug therapy, and which permit monitoring of patient response to such therapy.

### RELATED APPLICATION INFORMATION

This application is a continuation-in-part of U.S. Ser. No. 13/100,869, filed May 4, 2011, which claims the benefit of U.S. Application No. 61/332,545, filed on May 7, 2010, the contents of each of which are herein incorporated by reference.

### TECHNICAL FIELD

The present disclosure relates generally to the evaluation and/or treatment of a subject having or suspected of having a neoplastic condition, and in particular to the use of biomarkers for identifying patients receptive to a certain drug therapy, and which permit monitoring of patient response to such therapy.

### BACKGROUND OF THE INVENTION

Genetic heterogeneity of cancer is a factor complicating the development of efficacious cancer drugs. Cancers that are considered to be a single disease entity according to classical histopathological classification often reveal multiple genomic subtypes when subjected to molecular profiling. In certain cases, different genomic subtypes appear to have functional relevance to the efficacy of certain drugs. For example, the efficacy of certain targeted cancer drugs has been correlated with the presence of a genomic feature, such as a gene amplification. (See, e.g., T. J. Lynch et al., “Activating mutations in the epidermal growth factor receptor underlying responsiveness of non-small-cell lung cancer to gefitinib”, N. Engl. J. Med., 350: 2129-2139, 2004.)

Clinical studies have also identified certain plasma and serum markers that can be used to sub-classify lung cancer patients. The National Association of Clinical Biochemistry has published practice guidelines and recommendations for use of tumor markers in the clinic. (See The NACB Practice Guidelines and Recommendations for Use of Tumor Markers in the Clinic. Lung Cancer Section 3P). A pattern of tumor marker release has been correlated to the histological background of the tumor and can reveal mixed histological components. Table 1 summarizes the correlation of the markers CYFRA 21-1, CEA, NSE, and CA125 with tumor histology.

TABLE 1

| Histology | Before therapy | Post-therapy follow-up |
|---|---|---|
| Unknown | CYFRA 21-1, CEA, | After surgery: fol- |
| | NSE, ProGRP | lowing histology |
| | | In advanced disease: |
| | | using the leading marker |
| Adenocarcinoma | CYFRA 21-1 and CEA | CYFRA 21-1 and/or CEA |
| Squamous cell | CYFRA 21-1 and CEA | CYFRA 21-1 and/or CEA |
| carcinoma | (and SCCA) | (and/or SCCA) |
| Large cell | CYFRA 21-1 and CEA | CYFRA 21-1 and/or CEA |
| carcinoma | | |
| Small cell | NSE and ProGRP | NSE and/or ProGRP |
| carcinoma | | |

CEA, carcino embryonic antigen; CYFRA 21-1, cytokeratin 19 fragments; NSE, neuron specific enolase; ProGRP, progastrin-releasing peptide; SCCA, squamous cell carcinoma antigen

While the correlation of certain markers with certain subclasses of lung cancer may be helpful for distinguishing among different histological subtypes, the functional significance of such markers is generally not well understood.

ABT-869 (Linifanib) [N-(4-(3-amino-1H-indazol-4-yl)phenyl)-N′-(2-fluoro-5-methylphenyl)urea]), is a multi-targeted receptor tyrosine kinase inhibitor that has been shown to inhibit of all members of the VEGF and PDGF receptor families (e.g., KDR IC₅₀ value of 4 nM), and have less activity (IC₅₀ values>1 μM) against unrelated receptor tyrosine kinases, soluble tyrosine kinases and serine/threonine kinases. In addition, it exhibits potent anti-proliferative and apoptotic effects on tumor cells that are dependent on mutant, constitutively active, FLT3 and KIT kinases. Despite its potent anti-tumor activity, many malignant cell types are refractory to ABT-869. The cause of resistance is unknown.

Because of the potential therapeutic use of ABT-869 against various cancers, companion diagnostic assays that would identify those patients most receptive to ABT-869 therapy are needed. Additionally, a need exists for diagnostic methods that can be used to monitor the efficacy of therapy with ABT-869. A further need exist for companion assays using markers that can be measured in readily obtainable tissue samples such as blood or a blood plasma fraction.

### SUMMARY OF THE INVENTION

In one embodiment, the levels of the markers neuron-specific enolase (NSE), serum-soluble fragments of cytokeratin 19 (CYFRA 21-1), cancer antigen 125 (CA125) and carcinoma embryonic antigen (CEA) have been found to be indicative of the sensitivity of a subject's cancer to the administration of the drug ABT-869. Methods and kits described herein are based in part on the finding that any one or more of: a level of NSE below a predetermined level for NSE, a level of CA125 below a predetermined level for CA125, a level of CEA above a predetermined level for CEA, and a level of CYFRA 21-1 below a predetermined level for CYFRA 21-1, or any combination thereof, indicates increased sensitivity of the subject's cancer to the administration of ABT-869, relative to a subject that does not have a comparable level for any of the markers.

Accordingly, in one aspect, the present disclosure provides a method for predicting the sensitivity of a cancer in a subject to administration of ABT-869 to the subject, the method comprising the step of: determining in a sample obtained from the subject a level of at least one marker selected from the group consisting of: neuron-specific enolase (NSE), serum-soluble fragments of cytokeratin 19 (CYFRA 21-1), cancer antigen 125 (CA125) and carcinoma embryonic antigen (CEA), wherein any one of: a level of NSE below a predetermined level for NSE, a level of CA125 below a predetermined level for CA125, a level of CEA above a predetermined level for CEA, a level of CYFRA 21-1 below a predetermined level for CYFRA 21-1 or any combination thereof, indicates increased sensitivity of the subject's cancer to the administration of ABT-869 relative to a subject with a level of NSE, CYFRA 21-1 or CA125 above the predetermined level for each marker, or to a subject with a level of CEA below the predetermined level for each marker. The cancer may be non small-cell lung cancer. The method can comprise, for example, determining the levels of at least two markers selected from the group consisting of: NSE, CA125, CYFRA21-1 and CEA. The method can comprise determining the levels of NSE, CA125, CYFRA21-1 and CEA. The method may further comprise, for example, generating a marker signature for the subject from the levels of the two or more markers, wherein a marker signature having a predetermined pattern indicates an increased sensitivity, of the subject to administration of ABT-869, relative to a marker signature lacking the predetermined pattern. The method may further comprise comparing the levels of the two or more markers in the sample with levels of the same markers in a control sample by applying a classification tree analysis. The classification tree analysis may be performed by a computer process.

In another aspect, the present disclosure provides a method of predicting the sensitivity of a cancer in a subject to administration of ABT-869, the method comprising the step of: determining in a sample obtained from the subject levels of markers in a marker panel comprising NSE, CA125, CYFRA 21-1 and CEA, and comparing the level of each marker in the sample to a predetermined level for each marker, wherein the level of each marker in the sample relative to the predetermined level for each marker indicates sensitivity of the cancer to administration of ABT-869 to the subject. In the method, comparing the level of each marker in the sample to a predetermined level for each marker comprises comparing the marker levels to a level of each of the markers in a reference sample, wherein the reference sample contains each of the markers at a level corresponding to the predetermined level for each marker. The cancer can be non small-cell lung cancer. In the method, the NSE level in the subject's sample can be, for example, below the predetermined level for NSE, the CA125 level in the subject's sample can be below the predetermined level for CA125, the CYFRA 21-1 level in the subject's sample can be below the predetermined level for CYFRA 21-1, or the CEA level in the subject's sample is above the predetermined level for CEA, or any combination of all four conditions may be present. The method may further comprise generating a marker signature for the subject from the levels of the one or more markers, wherein a marker signature having a predetermined pattern indicates an increased sensitivity of the subject to administration of ABT-869, relative to a subject having a marker signature lacking the predetermined pattern. The method may further comprise comparing the levels of the markers in the subject's sample with levels of the markers in the reference sample by applying a classification tree analysis. The classification tree analysis may be performed, for example, by a computer process.

In another aspect, the present disclosure provides a method for classifying one or more subjects each having or suspected of having a cancer, for predicted efficacy of administration of ABT-869 for the treatment of the cancer, the method comprising determining in a sample from each subject, the level of at least one marker selected from the group consisting of: NSE, CYFRA 21-1, CA125 and CEA, wherein any one of: a reduced level of NSE relative to the level of NSE in a reference sample, a reduced level of CA125 relative to the level of CA125 in the reference sample, a reduced level of CYFRA 21-1 relative to the level of CYFRA 21-1 in the reference sample, an elevated level of CEA relative to the level CEA in the reference sample, or any combination thereof, indicates sensitivity of the cancer to administration of ABT-869 to the subject. The method may further comprise classifying each subject as being sensitive to treatment with ABT-869 based on the level of at least one of NSE, CA125, CYFRA 21-1 and CEA. In the method, the subject or subjects may have or may be suspected of having non small-cell lung cancer. According to the method, for example, the NSE level in the subject's sample can be reduced relative to the level of NSE in the reference sample. The CA 125 level in the subject's sample can be reduced relative to the level of CA125 in the reference sample. The CYFRA 21-1 level in the subject's sample can be reduced relative to the level of CYFRA 21-1 in the reference sample. The CEA level in the subject's sample can be elevated relative to the level of CEA in the reference sample. The method may further comprise generating a marker signature for each subject from the levels of the one or more markers, wherein a marker signature having a predetermined pattern indicates an increased sensitivity of the subject to administration of ABT-869, relative to a subject having a marker signature lacking the predetermined pattern. The method may further comprise comparing the levels of the markers in each subject's sample with levels of the same markers in the reference sample by applying a classification tree analysis, which may be performed by a computer process. In any of the above methods, the sample can be, a blood sample, including a serum or a plasma sample. Any of the above methods may further comprise the step of obtaining the sample from the subject or subject. In any of the above methods, the level of each marker can be determined for example by immunohistochemistry or immunoassay.

In another aspect, the present disclosure provides a kit for predicting the sensitivity of a cancer in a subject to administration of ABT-869 to the subject, the method comprising: a) an array comprising one or more binding reagents, each binding reagent having independent binding specificity for at least one marker selected from the group consisting of NSE, CA125, CYFRA 21-1 and CEA, wherein each binding reagent is independently bound to a discrete location on at least one substrate; and b) a control sample containing a predetermined level of the marker or markers in the array, wherein the predetermined level for each marker is a level relative to which a level for that marker indicates a sensitivity of the subject's cancer to the administration of ABT-869. The cancer for which the kit is configured to predict the sensitivity of administration of ABT-869 can be non small-cell lung cancer. In the kit, the level of NSE in the control sample can be, for example, a level below which a level of NSE in a subject's sample is indicative of sensitivity of the subject's cancer to the administration of ABT-869. The level of CA125 in the control sample can be a level below which a level of CA125 in a subject's sample is indicative of sensitivity of the subject's cancer to the administration of ABT-869. The level of CYFRA 21-1 in the control sample can be a level below which a level of CYFRA 21-1 in a subject's sample is indicative of sensitivity of the subject's cancer to the administration of ABT-869. The level of CEA in the control sample can be a level above which a level of CEA in a subject's sample is indicative of sensitivity of the subject's cancer to the administration of ABT-869. In the kit, the one or more substrates may each comprise a solid support coupled to a detectable label. The detectable label can comprise, for example, a fluorescent compound. The kit may further comprise instructions for determining the level of each marker in a sample from the subject. The subject's sample can be a blood sample, including a plasma sample or a serum sample.

In another aspect, the present disclosure provides a kit for predicting the sensitivity of a cancer in a subject to administration of ABT-869 to the subject, comprising: a) a microarray of markers comprising one or more selected from the group consisting of NSE, CA125, CYFRA 21-1, CEA and truncated forms thereof, and b) a control sample containing a predetermined level of the marker or markers, wherein the predetermined level for each marker is a level relative to which a level for that marker indicates a sensitivity of the subject's cancer to the administration of ABT-869. The cancer for which the kit is configured to predict the sensitivity of administration of ABT-869 can be non small-cell lung cancer. In the kit, the level of NSE in the control sample can be, for example, a level below which a level of NSE in a subject's sample is indicative of sensitivity of the subject's cancer to the administration of ABT-869. The level of CA125 in the control sample can be a level below which a level of CA125 in a subject's sample is indicative of sensitivity of the subject's cancer to the administration of ABT-869. The level of CYFRA 21-1 in the control sample can be a level below which a level of CYFRA 21-1 in a subject's sample is indicative of sensitivity of the subject's cancer to the administration of ABT-869. The level of CEA in the control sample can be a level above which a level of CEA in a subject's sample is indicative of sensitivity of the subject's cancer to the administration of ABT-869. In the kit, the one or more substrates may each comprise a solid support coupled to a detectable label. The detectable label can comprise, for example, a fluorescent compound. The kit may further comprise instructions for determining the level of each marker in a sample from the subject. The subject's sample can be a blood sample, including a plasma sample or a serum sample.

In another embodiment embodiment, the levels of the markers neuron-specific enolase (NSE), serum-soluble fragments of cytokeratin 19 (CYFRA 21-1), cancer antigen 125 (CA125) and pro-gastrin-releasing peptide (proGRP) have been found to be indicative of the sensitivity of a subject's cancer to the administration of the drug ABT-869. Methods and kits described herein are based in part on the finding that any one or more of: a level of NSE below a predetermined level for NSE, a level of CA125 below a predetermined level for CA125, a level of proGRP above a predetermined level for proGRP, and a level of CYFRA 21-1 below a predetermined level for CYFRA 21-1, or any combination thereof, indicates increased sensitivity of the subject's cancer to the administration of ABT-869, relative to a subject that does not have a comparable level for any of the markers.

Accordingly, in one aspect, the present disclosure provides a method for predicting the sensitivity of a cancer in a subject to administration of ABT-869 to the subject, the method comprising the step of: determining in a sample obtained from the subject a level of at least one marker selected from the group consisting of: neuron-specific enolase (NSE), serum-soluble fragments of cytokeratin 19 (CYFRA 21-1), cancer antigen 125 (CA125) and pro-gastrin-releasing peptide (proGRP), wherein any one of: a level of NSE below a predetermined level for NSE, a level of CA125 below a predetermined level for CA125, a level of proGRP above a predetermined level for proGRP, a level of CYFRA 21-1 below a predetermined level for CYFRA 21-1 or any combination thereof, indicates increased sensitivity of the subject's cancer to the administration of ABT-869 relative to a subject with a level of NSE, CYFRA 21-1 or CA125 above the predetermined level for each marker, or to a subject with a level of proGRP below the predetermined level for each marker. The cancer may be non small-cell lung cancer. The method can comprise, for example, determining the levels of at least two markers selected from the group consisting of: NSE, CA125, CYFRA21-1 and proGRP. The method can comprise determining the levels of NSE, CA125, CYFRA21-1 and proGRP. The method may further comprise, for example, generating a marker signature for the subject from the levels of the two or more markers, wherein a marker signature having a predetermined pattern indicates an increased sensitivity of the subject to administration of ABT-869, relative to a marker signature lacking the predetermined pattern. The method may further comprise comparing the levels of the two or more markers in the sample with levels of the same markers in a control sample by applying a classification tree analysis. The classification tree analysis may be performed by a computer process.

In another aspect, the present disclosure provides a method of predicting the sensitivity of a cancer in a subject to administration of ABT-869, the method comprising the step of: determining in a sample obtained from the subject levels of markers in a marker panel comprising NSE, CA125, CYFRA 21-1 and proGRP, and comparing the level of each marker in the sample to a predetermined level for each marker, wherein the level of each marker in the sample relative to the predetermined level for each marker indicates sensitivity of the cancer to administration of ABT-869 to the subject. In the method, comparing the level of each marker in the sample to a predetermined level for each marker comprises comparing the marker levels to a level of each of the markers in a reference sample, wherein the reference sample contains each of the markers at a level corresponding to the predetermined level for each marker. The cancer can be non small-cell lung cancer. In the method, the NSE level in the subject's sample can be, for example, below the predetermined level for NSE, the CA125 level in the subject's sample can be below the predetermined level for CA125, the CYFRA 21-1 level in the subject's sample can be below the predetermined level for CYFRA 21-1, or the proGRP level in the subject's sample is above the predetermined level for proGRP, or any combination of all four conditions may be present. The method may further comprise generating a marker signature for the subject from the levels of the one or more markers, wherein a marker signature having a predetermined pattern indicates an increased sensitivity of the subject to administration of ABT-869, relative to a subject having a marker signature lacking the predetermined pattern. The method may further comprise comparing the levels of the markers in the subject's sample with levels of the markers in the reference sample by applying a classification tree analysis. The classification tree analysis may be performed, for example, by a computer process.

In another aspect, the present disclosure provides a method for classifying one or more subjects each having or suspected of having a cancer, for predicted efficacy of administration of ABT-869 for the treatment of the cancer, the method comprising determining in a sample from each subject, the level of at least one marker selected from the group consisting of: NSE, CYFRA 21-1, CA125 and proGRP, wherein any one of: a reduced level of NSE relative to the level of NSE in a reference sample, a reduced level of CA125 relative to the level of CA125 in the reference sample, a reduced level of CYFRA 21-1 relative to the level of CYFRA 21-1 in the reference sample, an elevated level of CEA relative to the level CEA in the reference sample, or any combination thereof, indicates sensitivity of the cancer to administration of ABT-869 to the subject. The method may further comprise classifying each subject as being sensitive to treatment with ABT-869 based on the level of at least one of NSE, CA125, CYFRA 21-1 and proGRP. In the method, the subject or subjects may have or may be suspected of having non small-cell lung cancer. According to the method, for example, the NSE level in the subject's sample can be reduced relative to the level of NSE in the reference sample. The CA125 level in the subject's sample can be reduced relative to the level of CA125 in the reference sample. The CYFRA 21-1 level in the subject's sample can be reduced relative to the level of CYFRA 21-1 in the reference sample. The proGRP level in the subject's sample can be elevated relative to the level of proGRP in the reference sample. The method may further comprise generating a marker signature for each subject from the levels of the one or more markers, wherein a marker signature having a predetermined pattern indicates an increased sensitivity of the subject to administration of ABT-869, relative to a subject having a marker signature lacking the predetermined pattern. The method may further comprise comparing the levels of the markers in each subject's sample with levels of the same markers in the reference sample by applying a classification tree analysis, which may be performed by a computer process. In any of the above methods, the sample can be, a blood sample, including a serum or a plasma sample. Any of the above methods may further comprise the step of obtaining the sample from the subject or subject. In any of the above methods, the level of each marker can be determined for example by immunohistochemistry or immunoassay.

In another aspect, the present disclosure provides a kit for predicting the sensitivity of a cancer in a subject to administration of ABT-869 to the subject, the method comprising: a) an array comprising one or more binding reagents, each binding reagent having independent binding specificity for at least one marker selected from the group consisting of NSE, CA125, CYFRA 21-1 and proGRP, wherein each binding reagent is independently bound to a discrete location on at least one substrate; and b) a control sample containing a predetermined level of the marker or markers in the array, wherein the predetermined level for each marker is a level relative to which a level for that marker indicates a sensitivity of the subject's cancer to the administration of ABT-869. The cancer for which the kit is configured to predict the sensitivity of administration of ABT-869 can be non small-cell lung cancer. In the kit, the level of NSE in the control sample can be, for example, a level below which a level of NSE in a subject's sample is indicative of sensitivity of the subject's cancer to the administration of ABT-869. The level of CA125 in the control sample can be a level below which a level of CA125 in a subject's sample is indicative of sensitivity of the subject's cancer to the administration of ABT-869. The level of CYFRA 21-1 in the control sample can be a level below which a level of CYFRA 21-1 in a subject's sample is indicative of sensitivity of the subject's cancer to the administration of ABT-869. The level of CEA in the control sample can be a level above which a level of proGRP in a subject's sample is indicative of sensitivity of the subject's cancer to the administration of ABT-869. In the kit, the one or more substrates may each comprise a solid support coupled to a detectable label. The detectable label can comprise, for example, a fluorescent compound. The kit may further comprise instructions for determining the level of each marker in a sample from the subject. The subject's sample can be a blood sample, including a plasma sample or a serum sample.

In another aspect, the present disclosure provides a kit for predicting the sensitivity of a cancer in a subject to administration of ABT-869 to the subject, comprising: a) a microarray of markers comprising one or more selected from the group consisting of NSE, CA125, CYFRA 21-1, proGRP and truncated forms thereof, and b) a control sample containing a predetermined level of the marker or markers, wherein the predetermined level for each marker is a level relative to which a level for that marker indicates a sensitivity of the subject's cancer to the administration of ABT-869. The cancer for which the kit is configured to predict the sensitivity of administration of ABT-869 can be non small-cell lung cancer. In the kit, the level of NSE in the control sample can be, for example, a level below which a level of NSE in a subject's sample is indicative of sensitivity of the subject's cancer to the administration of ABT-869. The level of CA125 in the control sample can be a level below which a level of CA125 in a subject's sample is indicative of sensitivity of the subject's cancer to the administration of ABT-869. The level of CYFRA 21-1 in the control sample can be a level below which a level of CYFRA 21-1 in a subject's sample is indicative of sensitivity of the subject's cancer to the administration of ABT-869. The level of proGRP in the control sample can be a level above which a level of proGRP in a subject's sample is indicative of sensitivity of the subject's cancer to the administration of ABT-869. In the kit, the one or more substrates may each comprise a solid support coupled to a detectable label. The detectable label can comprise, for example, a fluorescent compound. The kit may further comprise instructions for determining the level of each marker in a sample from the subject. The subject's sample can be a blood sample, including a plasma sample or a serum sample.

### BRIEF DESCRIPTION OF THE DRAWINGS

FIG. 1 shows a Kaplan Meier plot showing the Overall Survival (OS) in days for stage 3/4 NSCLC patients treated with ABT-869 (M06-880) or other treatments.

FIG. 2 shows a Kaplan Meier plot showing the Overall Survival (OS) in days for stage 3/4 NSCLC patients treated with ABT-869 (M06-880) (FIG. 2A, left panel), or other treatments (FIG. 2B, right panel) segregated by signature negative or positive (NSE/CEA/Cyfra/CA 125).

FIG. 3 shows a Kaplan Meier plot showing the Overall Survival (OS) in days for stage 3/4 NSCLC patients treated with ABT-869 (M06-880) (FIG. 3A, left panel), or other treatments (FIG. 3B, right panel) segregated by signature negative or positive (NSE/pro-GPR/Cyfra/CA 125).

### DETAILED DESCRIPTION

### A. Definitions

Section headings as used in this section and the entire disclosure herein are not intended to be limiting.

a) As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

b) Neuron-Specific Enolase (“NSE”)

As used interchangeably herein, the terms “neurons-specific enolase” and “NSE” refer to a protein encoded by the human gene also known as enolase 2 (official symbol ENO2), and conservative variants thereof. As used herein, the term “official symbol” refers to that used in the EntrezGene database maintained by the United States National Center for Biotechnology Information.

c) Cancer Antigen 125 (“CA125”)

As used interchangeably herein, the terms “Cancer antigen 125” and “CA 125” refer to a carbohydrate antigen recognized as a tumor marker for ovarian cancer, and derived from Mucin 16, cell surface associated, also known as MUC16, which is a protein encoded by the human MUC16 gene (official symbol MUC16), and conservative variants of CA125.

d) Serum-Soluble Fragments of Cytokeratin 19 (“CYFRA 21-1”)

As used interchangeably herein, the terms “Serum-soluble fragments of cytokeratin 19” and “CYFRA 21-1” refer to an antigen recognized as a tumor marker for multiple cancers including lung cancer, and derived from cytokeratin 19, which is a protein encoded by the human keratin 19 gene (official symbol KRT19), and conservative variants of KRT19.

e) Carcinoembryonic Antigen (“CEA”) As used interchangeably herein, the terms “Carcinoembryonic antigen” and “CEA” refer to the human protein having the amino acid sequence under GenBank Accession No. CAE75559, and conservative variants thereof.

f) Pro-Gastrin-Releasing Peptide (“proGRP”)

As used interchangeably herein, the terms “pro-gastrin-releasing peptide” and “proGRP” refer to the stable precursor of gastrin-releasing peptide. Specifically, gastrin-releasing peptide is a member of the bombesin-like family of gastrin-releasing peptides. Its preproprotein, following cleavage of a signal peptide, is further processed to produce either the 27 aa gastrin-releasing peptide or the 10 aa neuromedin C.

g) Detectable Label

As used herein the term “detectable label” refers to any moiety that generates a measurable signal via optical, electrical, or other physical indication of a change of state of a molecule or molecules coupled to the moiety. Such physical indicators encompass spectroscopic, photochemical, biochemical, immunochemical, electromagnetic, radiochemical, and chemical means, such as but not limited to fluorescence, chemifluorescence, chemiluminescence, and the like.

h) Subject

As used herein, the terms “subject” and “patient” are used interchangeably irrespective of whether the subject has or is currently undergoing any form of treatment. As used herein, the terms “subject” and “subjects” refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc) and a human). Preferably, the subject is a human.

i) Test Sample

As used herein, the term “test sample” generally refers to a biological material being tested for and/or suspected of containing one or more cancer markers. The biological material may be derived from any biological source. Examples of biological materials include, but are not limited to, a peripheral blood sample, a tumor or suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a serum or a plasma fraction of a blood sample. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. If such methods of pretreatment are employed with respect to the test sample, such pretreatment methods are such that cancer cells remain in the test sample

### B. Markers Predictive of Cancer Sensitivity to ABT-869

The presently disclosed methods and kits are based in part on the surprising finding that levels of certain markers (or “biomarkers”) found in a test sample obtained from a subject are predictive of the sensitivity of the subject's cancer to administration of ABT-869. In one embodiment, these predictive markers include NSE, CA125, CYFRA 21-1 and CEA. In another embodiment, these predictive markers include NSE, CA125, CYRFRA 21-1 and proGRP.

The inventive methods are particularly useful with the compound ABT-869 (Linifanib; [N-(4-(3-amino-1H-indazol-4-yl)phenyl)-N′-(2-fluoro-5-methylphenyl)urea]), which is an ATP-competitive receptor tyrosine kinase (RTK) inhibitor that is a potent inhibitor of members of the vascular endothelial growth factor (VEGF) and platelet-derived growth factor (PDGF) receptor families. See Shankar D. B. et al., Blood, April 15: 109(8), 3400-8 (2007). The chemical structure of ABT-869 is:

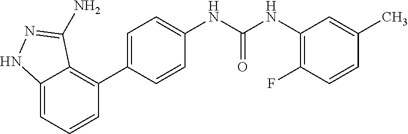

Other synthetic methods for ABT-869 have been described (see, e.g., A. Kruger et al., Org. Process Res. Dev. 13 (6), 1419-25 (2009)). Pharmaceutical compositions containing ABT-869 and routes and methods of its administration for cancer therapy are known and described in detail, for example, in U.S. patent application Ser. No. 11/636,189 (US 2007/0135387), the entire disclosure of which is hereby incorporated by reference.

A predictive marker is any marker that can be found and measured in a test sample from a subject, such as a blood sample which may be a plasma or a serum sample, the level (i.e. amount) of which marker in the sample is correlated with response of a cancer to a specific therapeutic compound and/or class of compounds. As described herein, the markers NSE, CA125, CYFRA 21-1 and CEA and NSE, CA125, CYFRA 21-1 and proGRP have been found to be predictive of a subject's sensitivity, or rather more specifically, the sensitivity of a subject's cancer, to treatment with ABT-869 by which is meant administration of ABT-869. To determine correlations of markers with clinical outcome, and more specifically with sensitivity to ABT-869, marker concentrations in subjects having a particular cancer of interest are measured for example at a starting time point for a baseline measure, and then at a second time point at about three weeks, for example at day 21 or 22 following initiation of a treatment regimen. Marker thresholds or “cut-offs” can be established for example as the median for the particular cancer type, or by using any other statistical approach by which such a cut-off value within a distribution of values may be selected. For each marker, subjects are categorized as having a marker level above or below the threshold. Survival, as for example Overall Survival, is then determined as a function of treatment class, and compared for each marker and treatment.

Thus, for each marker, a predetermined cut-off level is identified and provides a reference level that can then be used according to the methods and kits described herein. More specifically, as described elsewhere herein, a level of NSE below a predetermined level for NSE, a level of CYFRA 21-1 below a predetermined level for CYFRA 21-1, a level of CA125 below a predetermined level for CA 125, a level of CEA above a predetermined level for CEA, a level of proGRP above a predetermined level for proGRP or any combination thereof, indicates increased sensitivity of the subject's cancer to the administration of ABT-869, relative to a subject with a level of NSE, CA 125 or CYFRA 21-1 above the predetermined level for each marker, or to a subject with a level of CEA below the predetermined level for CEA or to a subject with a level of proGRP below the predetermined level for proGRP.

Typically the level of each marker in the test sample from the subject is determined using an immunohistochemistry or immunoassay technique, such as for example an enzyme immunoassay (EIA), and for which kits are readily commerically available from a number of commercial suppliers. An exemplary microparticle enzyme immunoassay technology is the AXSYM® System available from Abbott Laboratories. The assay may involve a multiplex technique so the levels of two or more markers can be determined from the output of a single assay process. The marker level of any two or more of the NSE, CA125, CYFRA 21-1 and CEA or NSE, CA125, CYFRA 21-1 and proGRP in a test sample can be combined to produce a marker signature (sometimes referred to as a “biomarker profile”), which is characterized by a pattern composed of at least of the two or more marker levels. One exemplary such pattern is composed of, for example, a level of NSE below a predetermined cut-off for NSE, together with one or more of a level of CA125 below the predetermined cut-off for CA125, a level of CYFRA 21-1 below the predetermined cut-off for CYFRA 21-1, and a level of CEA above the predetermined cut-off for CEA. The marker signature may include the level of one or more markers other than NSE, CA125, CYFRA 21-1 and CEA. A second exemplary such pattern is composed of, for example, a level of NSE below a predetermined cut-off for NSE, together with one or more of a level of CA125 below the predetermined cut-off for CA125, a level of CYFRA 21-1 below the predetermined cut-off for CYFRA 21-1, and a level of proGRP above the predetermined cut-off for proGRP. The marker signature may include the level of one or more markers other than NSE, CA125, CYFRA 21-1 and proGRP. A marker signature having a predetermined pattern, i.e. satisfying certain criteria such as a cut-off criterion for each at least two markers, indicates an increased sensitivity of the subject to administration of ABT-869, relative to a marker signature lacking the predetermined pattern.

Use of these markers in the methods and kits of the present disclosure provides a basis for developing targeted cancer therapy using ABT-869. The methods can be especially useful, for example, as a basis for companion assays for ABT-869 therapy, which is administered to a subject either as monotherapy or as part of combination therapy with other chemotherapy, such as conventional chemotherapy. The methods can be performed in relation to any cancer type for which it is determined that the marker levels are predictive of sensitivity of the cancer to administration of ABT-869. An exemplary such cancer is any carcinoma, such as non-small cell lung cancer, or any solid tumor.

### C. Methods

Methods for predicting the sensitivity of a cancer in a subject to administration of ABT-869 to the subject involve determining the level of at least one of the predictive markers as described herein, i.e neuron-specific enolase (NSE), cancer antigen 125 (CA125), serum-soluble fragments of cytokeratin 19 (CYFRA 21-1), carcinoma embryonic antigen (CEA), pro-gastrin-releasing peptide (proGRP). Any one or more of: 1) a level of NSE below a predetermined level for NSE, 2) a level of CA125 below a predetermined level for CA125, CYFRA 21-1 below a predetermined level for CYFRA 21-1, 3) a level of CEA above a predetermined level for CEA and 4) a level of proGRP above a predetermined level for proGRP, or any combination thereof, indicates increased sensitivity of the subject's cancer to the administration of ABT-869 as compared to a subject having a level of NSE, CA125 or CYFRA 21-1 above the predetermined level for each marker, or to a subject with a level of CEA below the predetermined level for CEA or to a subject with a level of proGRP below the predetermined level for proGRP. The methods can, for example, include determining the level of all four of NSE, CA125, CYFRA 21-1 and CEA. Alternatively the methods can, for example, include determining the level of all four of NSE, CA125, CYFRA 21-1 and proGRP. Cancers addressed by the present disclosure encompass any cancer for which ABT-869 therapy is contemplated, and especially any solid tumor including breast tumors, and carcinomas including hepatocellular carcinoma, renal cell carcinoma, small cell and large cell carcinomas, and combinations thereof, and include for example non-small cell lung cancer (NSCLC).

A cancer or a subject (patient) may be described as sensitive to, or resistant to a selected therapeutic drug regimen including administration of ABT-869, based on the ability of the drug to kill cancer cells or decrease tumor size and/or reduce overall cancer growth or spread (metastasis). Cancer cells or tumors that are not sensitive are deemed resistant to a therapeutic regimen and are those that do not respond to the drug regimen, for example those in which the drug regimen fails to significantly decrease tumor size or slow tumor growth or spread. Cancer cells that are sensitive to the therapeutic regimen are those that do respond to the drug regimen, resulting in decreased tumor size and/or slowed tumor growth or spread, and thus also in an increase in overall survival (“OS”). Monitoring of a response to the drug regimen can be accomplished by numerous pathological, clinical and imaging methods such as those described elsewhere herein and as are generally well known in the medical field. For example, tumor size can be evaluated using any soft tissue imaging technique, such as ultrasound. It will also be understood that the methods can further involve obtaining the test sample from the subject using any tissue sampling technique including but not limited to blood draw and fingerstick, and tissue biopsy techniques including needle biopsy.

When the levels of two or more markers are determined, the method may further comprise generating a marker signature for the subject from the levels of the two or more markers. A marker signature may include for example the two or marker levels, wherein each level relative to a cut-off value for that marker defines a feature of the marker signature, and the features together form the signature. A signature sharing a predetermined pattern, i.e. a pattern that reflects marker levels each having a certain relationship relative to a cut-off value for each marker, indicates an increased sensitivity of the subject to administration of ABT-869, relative to a marker signature lacking the predetermined pattern. For example, a predetermined signature pattern indicative of increased sensitivity of the subject to administration of ABT-869 and based on marker levels for all of NSE, CA125, CYFRA 21-1 and CEA is a pattern characterized by 1) a level of NSE that is below a predetermined level for NSE, 2) a level of CYFRA 21-1 that is below a predetermined level for CYFRA 21-1, 3) a level of a level of CA125 that is below a predetermined level for CA125, and 3) a level of CEA that is above a predetermined level for CEA. Any signature having all of these pattern features is exemplary of a signature that is indicative of sensitivity of the subject to administration of ABT-869. Alternatively, a predetermined signature pattern indicative of increased sensitivity of the subject to administration of ABT-869 and based on marker levels for all of NSE, CA125, CYFRA 21-1 and proGRP is a pattern characterized by 1) a level of NSE that is below a predetermined level for NSE, 2) a level of CYFRA 21-1 that is below a predetermined level for CYFRA 21-1, 3) a level of a level of CA125 that is below a predetermined level for CA125, and 3) a level of proGRP that is above a predetermined level for proGRP. Any signature having all of these pattern features is exemplary of a signature that is indicative of sensitivity of the subject to administration of ABT-869.

Analysis of the marker levels may further involve comparing the levels of at least two markers with levels of the same markers in a control sample, which may be performed by applying a classification tree analysis. Classification tree analyses are generally well-known and can be readily applied to analysis of marker levels using a computer process. For example, a reference 3D contour plot can be generated that reflects the marker levels as described herein that correlate with sensitivity of a cancer to treatment with ABT-869. For any given subject, a comparable 3D plot can be generated and the plot compared to the reference 3D plot to determine whether the subject has a marker signature indicative of sensitivity of the subject to administration of ABT-869. Classification tree analyses are well-suited for analyzing marker levels because they are especially amenable to graphical display and are easy to interpret. It will however be understood that any computer-based application can be used that compares multiple marker levels from two different subjects, or from a reference sample and a subject, and provides an output that indicates sensitivity of a subject to administration of ABT-869 based on the methods described herein.

The methods can be used to classify one or more subjects, each subject having or suspected of having a cancer, for predicted efficacy of administration of ABT-869 for the treatment of the cancer in the subject. Such an approach involves determining, in a sample from each subject, the level of at least one of the markers NSE, CA125, CYFRA 21-1 and CEA or NSE, CA125, CYFRA 21-1 and proGRP and comparing the level of each marker to its level in a reference sample. The reference sample contains an amount of each marker that corresponds to predetermined cut-off value for the marker. Any one of: 1) a reduced level of NSE relative to the level of NSE in a reference sample, 2) a reduced level of CYFRA 21-1 relative to the level of CYFRA21-1 in the reference sample, 3) an elevated level of CEA relative to the level CEA in the reference sample, 4) a reduced level of CA 125 relative to the level CA 125 in the reference sample or any combination thereof, indicates sensitivity of the cancer to administration of ABT-869 to the subject. Alternatively one of: 1) a reduced level of NSE relative to the level of NSE in a reference sample, 2) a reduced level of CYFRA 21-1 relative to the level of CYFRA21-1 in the reference sample, 3) an elevated level of proGRP relative to the level proGRP in the reference sample, 4) a reduced level of CA125 relative to the level CA125 in the reference sample or any combination thereof, indicates sensitivity of the cancer to administration of ABT-869 to the subject. Thus the methods can be used for example to target a patient population in which treatment with ABT-869 is likely to produce superior results as compared to alternative therapies.

### D. Kits

The present disclosure also provides kits for predicting the sensitivity of a cancer in a subject to administration of ABT-869 to the subject. The kit can comprise for example an array of one or more binding reagents, and a control sample containing a predetermined level of the marker or markers, wherein the predetermined level for each marker is a level relative to which a level for that marker indicates a sensitivity of the subject's cancer to the administration of ABT-869. The predetermined level for each marker is for example a cut-off or threshold value determined according to a statistical analysis, for example as described elsewhere herein, such as in the Examples. Each binding reagent has independent binding specificity for at least one of NSE, CA125, CYFRA 21-1, and CEA or at least one of NSE, CA125, CYFRA 21-1 and proGRP. Exemplary such binding reagents are antibodies. Alternatively, a kit may include an array of two or more of the markers or truncated forms or fragments thereof.

### Antibodies

A binding reagent may be for example a polyclonal antibody, a monoclonal antibody, a chimeric antibody, a human antibody, an affinity maturated antibody or an antibody fragment. A sandwich immunoassay format may be used in which both a capture and a detection antibody are used for each marker. Antibodies may be bound, for example conjugated, to a detectable label. While monoclonal antibodies are highly specific to the marker/antigen, a polyclonal antibody can preferably be used as a capture antibody to immobilize as much of the marker/antigen as possible. A monoclonal antibody with inherently higher binding specificity for the marker/antigen may then preferably be used as a detection antibody for each marker/antigen. In any case, the capture and detection antibodies recognize non-overlapping epitopes on each marker, preferably without interfering with the binding of the other.

Polyclonal antibodies are raised by injecting (e.g., subcutaneous or intramuscular injection) an immunogen into a suitable non-human mammal (e.g., a mouse or a rabbit). Generally, the immunogen should induce production of high titers of antibody with relatively high affinity for the target antigen. If desired, the marker may be conjugated to a carrier protein by conjugation techniques that are well known in the art. Commonly used carriers include keyhole limpet hemocyanin (KLH), thyroglobulin, bovine serum albumin (BSA), and tetanus toxoid. The conjugate is then used to immunize the animal. The antibodies are then obtained from blood samples taken from the animal. The techniques used to produce polyclonal antibodies are extensively described in the literature (see, e.g., Methods of Enzymology, “Production of Antisera with Small Doses of Immunogen: Multiple Intradermal Injections,” Langone, et al. eds. (Acad. Press, 1981)). Polyclonal antibodies produced by the animals can be further purified, for example, by binding to and elution from a matrix to which the target antigen is bound. Those of skill in the art will know of various techniques common in the immunology arts for purification and/or concentration of polyclonal, as well as monoclonal, antibodies (see, e.g., Coligan, et al. (1991) Unit 9, Current Protocols in Immunology, Wiley Interscience).

For many applications, monoclonal antibodies (mAbs) are preferred. The general method used for production of hybridomas secreting mAbs is well known (Kohler and Milstein (1975) Nature, 256:495). Briefly, as described by Kohler and Milstein, the technique entailed isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody that bound to cancer cell lines. Confirmation of specificity among mAbs can be accomplished using routine screening techniques (such as the enzyme-linked immunosorbent assay, or “ELISA”) to determine the elementary reaction pattern of the mAb of interest.

As used herein, the term “antibody” also encompasses antigen-binding antibody fragments, e.g., single chain antibodies (scFv or others), which can be produced/selected using phage display technology. The ability to express antibody fragments on the surface of viruses that infect bacteria (bacteriophage or phage) makes it possible to isolate a single binding antibody fragment, e.g., from a library of greater than 1010 nonbinding clones. To express antibody fragments on the surface of phage (phage display), an antibody fragment gene is inserted into the gene encoding a phage surface protein (e.g., pIII) and the antibody fragment-pIII fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage-bearing antigen-binding antibody fragments can be separated from non-binding phage by antigen affinity chromatography (McCafferty et al. (1990) Nature, 348: 552-554). Depending on the affinity of the antibody fragment, enrichment factors of 20-fold-1,000,000-fold are obtained for a single round of affinity selection. By infecting bacteria with the eluted phage, however, more phage can be grown and subjected to another round of selection. In this way, an enrichment of 1000-fold in one round can become 1,000,000-fold in two rounds of selection (McCafferty et al. (1990) Nature, 348: 552-554). Thus, even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment, natural VH and VL repertoires present in human peripheral blood lymphocytes are isolated from unimmunized donors by PCR. The V-gene repertoires can be spliced together at random using PCR to create a scFv gene repertoire which can be cloned into a phage vector to create a library of 30 million phage antibodies (Id.). From a single “naive” phage antibody library, binding antibody fragments have been isolated against more than 17 different antigens, including haptens, polysaccharides, and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor, and CEA (Griffiths et al. (1993) EMBO J. 12: 725-734). The antibody fragments are highly specific for the antigen used for selection and have affinities in the 1 nM to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

As those of skill in the art readily appreciate, antibodies can be also prepared by any of a number of commercial services (e.g., Berkeley Antibody Laboratories, Bethyl Laboratories, Anawa, Eurogenetec, etc.).

### Solid Phase

In kits according to the present disclosure, each binding reagent may be bound to a solid phase. A solid phase can be any suitable material with sufficient surface affinity to bind an antibody, for example each capture antibody having a specific binding for one of the markers. The solid phase can take any of a number of forms, such as a magnetic particle, bead, test tube, microtiter plate, cuvette, membrane, a scaffolding molecule, quartz crystal, film, filter paper, disc or a chip. Useful solid phase materials include: natural polymeric carbohydrates and their synthetically modified, crosslinked, or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer. All of these materials may be used in suitable shapes, such as films, sheets, tubes, particulates, or plates, or they may be coated onto, bonded, or laminated to appropriate inert carriers, such as paper, glass, plastic films, fabrics, or the like. Nitrocellulose has excellent absorption and adsorption qualities for a wide variety of reagents including monoclonal antibodies. Nylon also possesses similar characteristics and also is suitable. Any of the above materials can be used to form an array, such as a microarray, of one or more specific binding reagents.

Alternatively, the solid phase can constitute microparticles. Microparticles useful in the present disclosure can be selected by one skilled in the art from any suitable type of particulate material and include those composed of polystyrene, polymethylacrylate, polypropylene, latex, polytetrafluoroethylene, polyacrylonitrile, polycarbonate, or similar materials. Further, the microparticles can be magnetic or paramagnetic microparticles, so as to facilitate manipulation of the microparticle within a magnetic field. In an exemplary embodiment the microparticles are carboxylated magnetic microparticles. Microparticles can be suspended in the mixture of soluble reagents and test sample or can be retained and immobilized by a support material. In the latter case, the microparticles on or in the support material are not capable of substantial movement to positions elsewhere within the support material. Alternatively, the microparticles can be separated from suspension in the mixture of soluble reagents and test sample by sedimentation or centrifugation. When the microparticles are magnetic or paramagnetic the microparticles can be separated from suspension in the mixture of soluble reagents and test sample by a magnetic field. The methods of the present disclosure can be adapted for use in systems that utilize microparticle technology including automated and semi-automated systems wherein the solid phase comprises a microparticle. Such systems include those described in pending U.S. App. No. 425,651 and U.S. Pat. No. 5,089,424, which correspond to published EPO App. Nos. EP 0 425 633 and EP 0 424 634, respectively, and U.S. Pat. No. 5,006,309.

Other considerations affecting the choice of solid phase include the ability to minimize non-specific binding of labeled entities and compatibility with the labeling system employed. For, example, solid phases used with fluorescent labels should have sufficiently low background fluorescence to allow signal detection. Following attachment of a specific capture antibody, the surface of the solid support may be further treated with materials such as serum, proteins, or other blocking agents to minimize non-specific binding.

### Detection Systems

Kits according to the present disclosure may include one or more detectable labels. The one or more specific binding reagents, e.g. antibodies, may be bound to a detectable label. Detectable labels suitable for use include any compound or composition having a moiety that is detectable by spectroscopic, photochemical, biochemical, immunochemical, electrical, optical, or chemical means. Such labels include, for example, an enzyme, oligonucleotide, nanoparticle chemiluminophore, fluorophore, fluorescence quencher, chemiluminescence quencher, or biotin. Thus for example, in an immunoassay kit configured to employ an optical signal, the optical signal is measured as an analyte concentration dependent change in chemiluminescence, fluorescence, phosphorescence, electrochemiluminescence, ultraviolet absorption, visible absorption, infrared absorption, refraction, surface plasmon resonance. In an immunoassay kit configured to employ an electrical signal, the electrical signal is measured as an analyte concentration dependent change in current, resistance, potential, mass to charge ratio, or ion count. In an immunoassay kit configured to employ a change-of-state signal, the change of state signal is measured as an analyte concentration dependent change in size, solubility, mass, or resonance.

Useful labels according to the present disclosure include magnetic beads (e.g., Dynabeads™), fluorescent dyes (e.g., fluorescein, Texas Red, rhodamine, green fluorescent protein) and the like (see, e.g., Molecular Probes, Eugene, Oreg., USA), chemiluminescent compounds such as acridinium (e.g., acridinium-9-carboxamide), phenanthridinium, dioxetanes, luminol and the like, radiolabels (e.g., 3H, 125I, 35S, 14C, or 32P), catalysts such as enzymes (e.g., horse radish peroxidase, alkaline phosphatase, beta-galactosidase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The label can be attached to each antibody, for example to a detection antibody in a sandwich immunoassay format, prior to, or during, or after contact with the biological sample. So-called “direct labels” are detectable labels that are directly attached to or incorporated into the antibody prior to use in the assay. Direct labels can be attached to or incorporated into the detection antibody by any of a number of means well known to those of skill in the art.

In contrast, so-called “indirect labels” typically bind to each antibody at some point during the assay. Often, the indirect label binds to a moiety that is attached to or incorporated into the detection agent prior to use. Thus, for example, each antibody can be biotinylated before use in an assay. During the assay, an avidin-conjugated fluorophore can bind the biotin-bearing detection agent, to provide a label that is easily detected.

In another example of indirect labeling, polypeptides capable of specifically binding immunoglobulin constant regions, such as polypeptide A or polypeptide G, can also be used as labels for detection antibodies. These polypeptides are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542). Such polypeptides can thus be labeled and added to the assay mixture, where they will bind to each capture and detection antibody, as well as to the autoantibodies, labeling all and providing a composite signal attributable to analyte and autoantibody present in the sample.

Some labels may require the use of an additional reagent(s) to produce a detectable signal. In an ELISA, for example, an enzyme label (e.g., beta-galactosidase) will require the addition of a substrate (e.g., X-gal) to produce a detectable signal. In an immunoassay kit configured to use an acridinium compound as the direct label, a basic solution and a source of hydrogen peroxide can also be included in the kit.

Test kits according to the present disclosure preferably include instructions for determining the level of each marker in a sample from the subject, for example by carrying out one or more immunoassays. The instructions may further include instructions for analyzing a test sample of a specific type, such as a blood sample, or more specifically a serum sample or a plasma sample. Instructions included in kits of the present disclosure can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### E. Adaptations of the Methods of the Present Disclosure

One skilled in the art would readily appreciate that the biomarkers, oligonucleotides, methods, kits and related compositions described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

### EXAMPLES

By way of example, and not of limitation, examples of the present disclosures shall now be given.

### Example 1

### General Strategy to Correlate Markers with Clinical Outcome

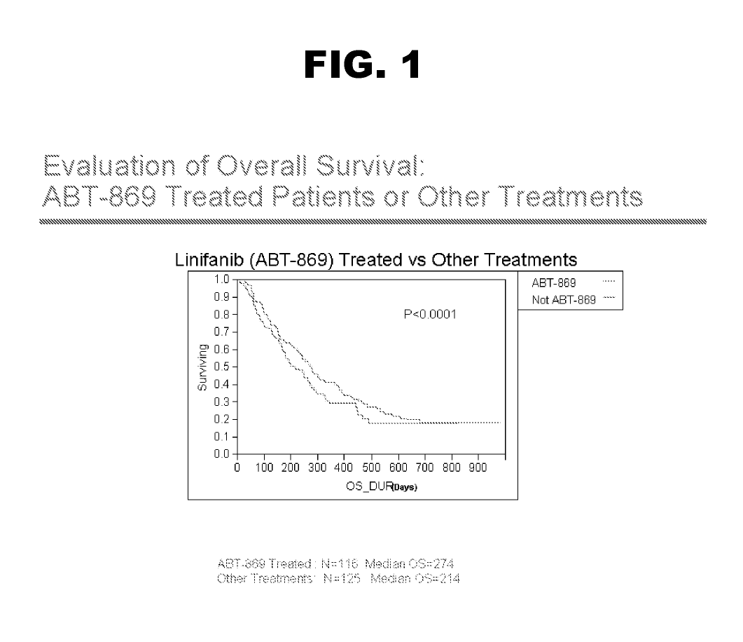

Marker concentrations were Measured by immunoassay at baseline in NSCLC trials. NSCLC subjects were assigned to one of two cohorts. Cohort one consisted of patients from the clinical trial M05-780. These patients received pemetrexed (available under the brand name Alimta® from Eli Lilly and Company, Indianapolis, Ind.), with or without ABT-751 ((N-[2-[(4-Hydroxyphenyl)amino]-3-pyridinyl]-4-methoxybenzenesulfonamide, available from Abbott Laboratories, Abbott Park, Ill.). Cohort two consisted of patients from the clinical trial M06-880. The patients in cohort two were treated with only ABT-869. All patients were diagnosed with stage 3/4 NSCLC. The patient overall survival of the cohorts is shown in FIG. 1.

Thresholds for each marker were established using multiple methods including but not limited to, median value determination, statistical modeling for optimal thresholds, values determined in the community to be predictive for NSCLC vs benign lung disease and comparison of relative concentration of the marker in patients with stable disease vs rapid progression on therapy with ABT-869. These thresholds were then used to classify each patient as having marker concentrations above or below the threshold. Survival as a function of classification was compared for each marker and treatment.

### Example 2

### Analysis of Data Across Multiple NSCLC Trials with Differing Therapeutics to Identify Predictive Markers

Each markers was measured for each patient before administration of therapy. Thresholds for each marker were established using multiple methods including but not limited to, median value determination, statistical modeling for optimal thresholds, values determined in the community to be predictive for NSCLC vs benign lung disease and comparison of relative concentration of the marker in patients with stable disease vs rapid progression on therapy with ABT-869. These thresholds were then used to classify each patient as having marker concentrations above or below the threshold. Survival as a function of classification was compared for each marker and treatment.

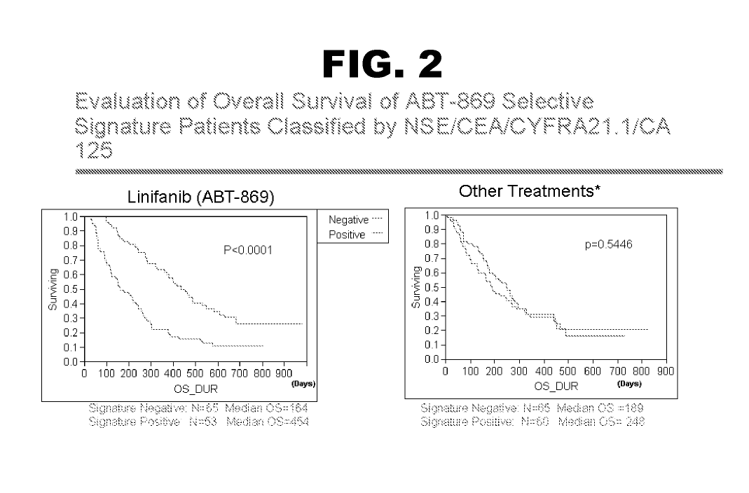

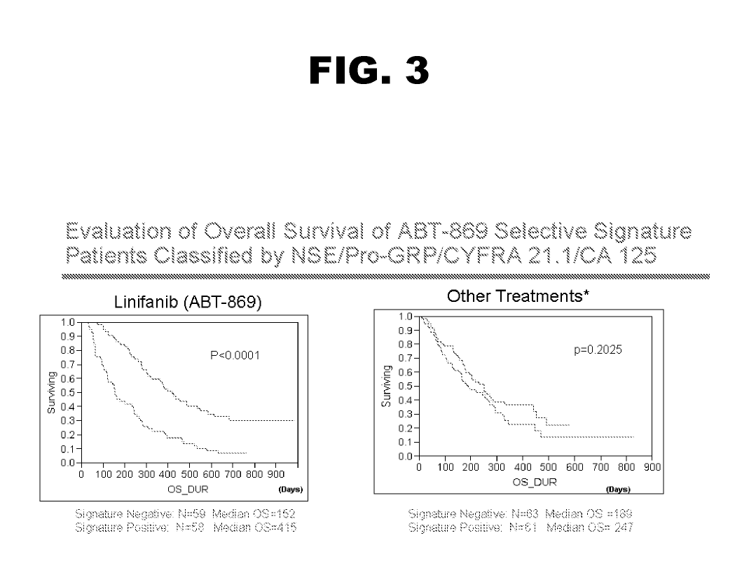

To identify biomarker-based patient classifications associated with response to a particular drug (predictive signatures), survival curves was compared across treatment groups. FIGS. 2 and 3 show the Kaplan-Meier analysis from two possible classifications. In FIG. 2, Group 1 (blue line) consists of those patients with NSE, CYFRA 21-1 and CA125 below the determined threshold for each marker, and CEA above the threshold for CEA. Group 2 patients (red line) were those with NSE, CYFRA 21-1 and CA125 above the threshold for each marker, and CEA below the threshold for CEA. The plot at left compares Overall Survival in patients that received ABT-869 (M06-880) whereas the plot on the right compares Overall Survival in patients treated with Alimta with or without ABT-751.

In FIG. 3, Group 1 (blue line) consists of those patients with NSE, CYFRA 21-1 and CA125 below the determined threshold for each marker, and CEA above the cut-off for CEA. Group 2 patients (red line) were those with NSE, CYFRA 21-1 and CA125 above the cut-off for each marker, and CEA below the cut-off for pro-GRP. The plot at left compares Overall Survival in patients that received ABT-869 (M06-880) whereas the plot on the right compares Overall Survival in patients treated with Alimta with or without ABT-751.

What is claimed is:

1. A method for predicting the sensitivity of a cancer in a subject to administration of ABT-869 to the subject, the method comprising the step of:
determining in a sample obtained from the subject a level of at least one marker selected from the group consisting of: neuron specific enolase (NSE), cancer antigen 125 (CA125), CYFRA 21-1, carcinoma embryonic antigen (CEA), and pro-gastrin-releasing peptide (proGRP), wherein any one of: a level of NSE below a predetermined level for NSE, a level of CYFRA 21-1 below a predetermined level for CYFRA 21-1, a level of CA125 below a predetermined level for CA125, a level of CEA above a predetermined level for CEA, a level of proGRP above a predetermined level for proGRP or any combination thereof, indicates increased sensitivity of the subject's cancer to the administration of ABT-869 relative to a subject with a level of NSE, CA125 or CYFRA 21-1 above the predetermined level for each marker, or to a subject with a level of CEA below the predetermined level for each marker or a subject with a level of proGRP below the predetermined level for each marker.

2. The method according to claim 1, wherein the cancer is non small-cell lung cancer.

3. The method according to claim 1, wherein the sample is a blood sample.

4. The method according to claim 1, wherein the sample is a serum or a plasma sample.

5. The method according to claim 1, wherein the method further comprises obtaining the sample from the subject.

6. The method according to claim 1, wherein the level of each marker is determined by immunohistochemistry or immunoassay.

7. The method according to claim 1, comprising determining the levels of at least two markers selected from the group consisting of: NSE, CYFRA 21-1, CA125, CEA and proGRP.

8. The method according to claim 1, comprising determining the levels of NSE, CA125, CYFRA 21-1 and CEA.

9. The method according to claim 1, comprising determining the levels of NSE, CA125, CYFRA 21-1 and proGRP.

10. The method according to claims 7, 8 or 9, wherein the method further comprises generating a marker signature for the subject from the levels of the two or more markers, wherein a marker signature having a predetermined pattern indicates an increased sensitivity of the subject to administration of ABT-869, relative to a marker signature lacking the predetermined pattern.

11. The method according to claims 7, 8 or 9, wherein the method further comprises comparing the levels of the two or more markers in the sample with levels of the same markers in a control sample by applying a classification tree analysis.

12. The method according to claim 11, wherein the classification tree analysis is performed by a computer process.

13. A method of predicting the sensitivity of a cancer in a subject to administration of ABT-869, the method comprising the step of:
determining in a sample obtained from the subject levels of markers in a marker panel comprising NSE, CA125, CYFRA 21-1 and CEA, and comparing the level of each marker in the sample to a predetermined level for each marker, wherein the level of each marker in the sample relative to the predetermined level for each marker indicates sensitivity of the cancer to administration of ABT-869 to the subject.

14. A method of predicting the sensitivity of a cancer in a subject to administration of ABT-869, the method comprising the step of:
determining in a sample obtained from the subject levels of markers in a marker panel comprising NSE, CA125, CYFRA 21-1 and proGRP, and comparing the level of each marker in the sample to a predetermined level for each marker, wherein the level of each marker in the sample relative to the predetermined level for each marker indicates sensitivity of the cancer to administration of ABT-869 to the subject.

15. The method according to claims 13 or 14, wherein comparing the level of each marker in the sample to a predetermined level for each marker comprises comparing the marker levels to a level of each of the markers in a reference sample, wherein the reference sample contains each of the markers at a level corresponding to the predetermined level for each marker.

16. The method according to claims 13 or 14, wherein the cancer is non small-cell lung cancer.

17. The method according to claims 13 or 14, wherein the NSE level in the subject's sample is below the predetermined level for NSE.

18. The method according to claims 13 or 14, wherein the CYFRA 21-1 level in the subject's sample is below the predetermined level for CYFRA 21-1.

19. The method according to claim 13, wherein the CEA level in the subject's sample is above the predetermined level for CEA.

20. The method according to claim 14, wherein the proGRP level in the subject's sample is above the predetermined level for proGRP.

21. The method according to claims 13 or 14, wherein the CA125 level in the subject's sample is below the predetermined level for CA125.

22. The method according to claims 13 or 14, wherein the sample from the subject is a blood sample.

23. The method according to claims 13 or 14, wherein the sample from the subject is a serum or a plasma sample.

24. The method according to claims 13 or 14, wherein the method further comprises obtaining the sample from the subject.

25. The method according to claims 13 or 14, wherein the level of each marker in the subject's sample is determined by immunohistochemistry or immunoassay.

26. The method of claims 13 or 14, wherein the method further comprises generating a marker signature for the subject from the levels of the markers, wherein a marker signature having a predetermined pattern indicates an increased sensitivity of the subject to administration of ABT-869, relative to a subject having a marker signature lacking the predetermined pattern.

27. The method of claims 13 or 14, wherein the method further comprises comparing the levels of the markers in the subject's sample with levels of the markers in the reference sample by applying a classification tree analysis.

28. The method of claim 27, wherein the classification tree analysis is performed by a computer process.

29. A method for classifying one or more subjects each having or suspected of having a cancer, for predicted efficacy of administration of ABT-869 for the treatment of the cancer, the method comprising determining in a sample from each subject, the level of at least one marker selected from the group consisting of: NSE, CA125, CYFRA 21-1, CEA and proGRP, wherein any one of: a reduced level of NSE relative to the level of NSE in a reference sample, a reduced level of CYFRA 21-1 relative to the level of CYFRA21-1 in the reference sample, an elevated level of CEA relative to the level CEA in the reference sample, an elevated level of proGRP relative to the level of proGRP in the reference sample, a reduced level of CA125 relative to the level CA125 in the reference sample or any combination thereof, indicates sensitivity of the cancer to administration of ABT-869 to the subject.

30. The method according to claim 29, wherein the method further comprises classifying each subject as being sensitive to treatment with ABT-869 based on the level of at least one of NSE, CYFRA 21-1, CA125 and CEA.

31. The method according to claim 29, wherein the subject or subjects have or are suspected of having non small-cell lung cancer.

32. The method according to claim 29, wherein the NSE level in the subject's sample is reduced relative to the level of NSE in the reference sample.

33. The method according to claim 29, wherein the CYFRA 21-1 level in the subject's sample is reduced relative to the level of CYFRA 21-1 in the reference sample.

34. The method according to claim 29, wherein the CEA level in the subject's sample is elevated relative to the level of CEA in the reference sample.

35. The method according to claim 29, wherein the proGRP level in the subject's sample is elevated relative to the level of proGRP in the reference sample.

36. The method according to claim 29, wherein the CA125 level in the subject's sample is reduced relative to the level of CA125 in the reference sample.

37. The method according to claim 29, wherein the sample is a blood sample.

38. The method according to claim 29, wherein the sample is a serum or a plasma sample.

39. The method according to claim 29, wherein the method further comprises obtaining the sample from each subject.

40. The method according to claim 29, wherein the level of each marker is determined by immunohistochemistry or immunoassay.

41. The method according to claim 29, wherein the method further comprises generating a marker signature for each subject from the levels of the one or more markers, wherein a marker signature having a predetermined pattern indicates an increased sensitivity of the subject to administration of ABT-869, relative to a subject having a marker signature lacking the predetermined pattern.

42. The method according to claim 29, wherein the method further comprises comparing the levels of the markers in each subject's sample with levels of the same markers in the reference sample by applying a classification tree analysis.

43. A kit for predicting the sensitivity of a cancer in a subject to administration of ABT-869 to the subject, comprising:
a. an array comprising one or more binding reagents, each binding reagent having independent binding specificity for at least one marker selected from the group consisting of NSE, CYFRA 21-1, CA125, CEA or proGRP, wherein each binding reagent is independently bound to a discrete location on at least one substrate; and
b. a control sample containing a predetermined level of the marker or markers in the array, wherein the predetermined level for each marker is a level relative to which a level for that marker indicates a sensitivity of the subject's cancer to the administration of ABT-869.

44. The kit according to claim 43, wherein the cancer is non small-cell lung cancer.

45. The kit according to claim 43, wherein the level of NSE in the control sample is a level below which a level of NSE in a subject's sample is indicative of sensitivity of the subject's cancer to the administration of ABT-869.

46. The kit according to claim 43, wherein the level of CYFRA 21-1 in the control sample is a level below which a level of CYFRA 21-1 in a subject's sample is indicative of sensitivity of the subject's cancer to the administration of ABT-869.

47. The kit according to claim 43, wherein the level of CA125 in the control sample is a level below which a level of CA125 in a subject's sample is indicative of sensitivity of the subject's cancer to the administration of ABT-869.

48. The kit according to claim 43, wherein the level of CEA in the control sample is a level above which a level of CEA in a subject's sample is indicative of sensitivity of the subject's cancer to the administration of ABT-869.

49. The kit according to claim 43, wherein the level of proGRP in the control sample is a level above which a level of proGRP in a subject's sample is indicative of sensitivity of the subject's cancer to the administration of ABT-869.

50. The kit according to claim 43, wherein the one or more substrates each comprise a solid support coupled to a detectable label.

51. The kit according to claim 50, wherein the detectable label comprises a fluorescent compound.

52. The kit according to claim 43, further comprising instructions for determining the level of each marker in a sample from the subject.

53. The kit according to claim 52, wherein the sample from the subject is a blood sample.

54. The kit according to claim 52, wherein the sample from the subject is a plasma sample.

55. The kit according to claim 52, wherein the sample from the subject is a serum sample.

56. A kit for predicting the sensitivity of a cancer in a subject to administration of ABT-869 to the subject, comprising:
a. a microarray of markers comprising one or more selected from the group consisting of NSE, CYFRA 21-1, CA125, CEA, proGRP and truncated forms thereof, and
b. a control sample containing a predetermined level of the marker or markers, wherein the predetermined level for each marker is a level relative to which a level for that marker indicates a sensitivity of the subject's cancer to the administration of ABT-869.

57. The kit according to claim 56, wherein the cancer is non small-cell lung cancer.

58. The kit according to claim 56, wherein the level of NSE in the control sample is a level below which a level of NSE in a subject's sample is indicative of sensitivity of the subject's cancer to the administration of ABT-869.

59. The kit according to claim 56, wherein the level of CYFRA 21-1 in the control sample is a level below which a level of CYFRA 21-1 in a subject's sample is indicative of sensitivity of the subject's cancer to the administration of ABT-869.

60. The kit according to claim 56, wherein the level of CA125 in the control sample is a level below which a level of CA125 in a subject's sample is indicative of sensitivity of the subject's cancer to the administration of ABT-869.

61. The kit according to claim 56, wherein the level of CEA in the control sample is a level above which a level of CEA in a subject's sample is indicative of sensitivity of the subject's cancer to the administration of ABT-869.

62. The kit according to claim 56, wherein the level of proGRP in the control sample is a level above which a level of proGRP in a subject's sample is indicative of sensitivity of the subject's cancer to the administration of ABT-869.

63. The kit according to claim 56, further comprising instructions for determining the level of each marker in a sample from the subject.

64. The kit according to claim 56, wherein the sample from the subject is a blood sample.

65. The kit according to claim 56, wherein the sample from the subject is a plasma sample.

66. The kit according to claim 56, wherein the sample from the subject is a serum sample.
